SEQUENCE LISTING (5 sequences)

SEQ ID NO: 1 - DNA, 10 bp, human
  oligonucleotide
ccctcccagc

SEQ ID NO: 2 - DNA, 10 bp, human
  oligonucleotide
gaagccctgg

SEQ ID NO: 3 - DNA, 12 bp, human
  oligonucleotide
aattcgcccacc

SEQ ID NO: 4 - DNA, 295 bp, human
  oligonucleotide
cgcgtgctagcccgggctcgagccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgaattcgcccaccatg

SEQ ID NO: 5 - DNA, 295 bp, human; identical to SEQ ID NO: 4
  oligonucleotide

(19) United States
(12) Patent Application Publication
(10) Pub. No.: US 2005/0250186 A1
(43) Pub. Date: Nov. 10, 2005

(54) Methods and compositions for modulating telomerase reverse transcriptase (TERT) expression

(76) Inventors: William H. Andrews, Reno, NV (US); Christopher Foster, Reno, NV (US); Stephanie Fraser, Sparks, NV (US); Hamid Mohammadpour, Reno, NV (US)

Correspondence Address: DLA PIPER RUDNICK GRAY CARY US LLP, 153 TOWNSEND STREET, SUITE 800, SAN FRANCISCO, CA, 94107-1907, US

(21) Appl. No.: 10/140,776

(22) Filed: May 7, 2002

Related U.S. Application Data
(60) Provisional application No. 60/289,717, filed on May 8, 2001.

(51) Int. Cl. C12P 21/06; C12P 19/34

(52) U.S. Cl. 435/91.1; 435/91.33; 435/69.1

(57) ABSTRACT

Nucleic acid compositions comprising a TF-13 or TF-8 repressor binding site that acts to repress transcription of the telomerase reverse transcriptase (TERT) coding sequence, as well as vectors and constructs including the same, are provided. Also provided are methods of modulating, e.g., inhibiting, the TERT transcription repressing activity of the subject TF-8 and TF-13 repressor binding site regions in order to regulate, e.g., enhance, telomerase expression, which methods find use in a variety of different applications, including the production of reagents for use in life science research, therapeutic applications and the like. In addition, methods of screening for agents that modulate the TERT transcription repressing activity of the subject TF-8 and TF-13 sites are provided. The subject invention finds use in, among other applications, the regulation of TERT expression.

### CROSS-REFERENCE TO RELATED APPLICATIONS

Pursuant to 35 U.S.C. § 119(e), this application claims priority to the filing dates of the U.S. Provisional Patent Application Ser. Nos. 60/289,717, filed May 8, 2001; the disclosure of which is herein incorporated by reference.

### INTRODUCTION

1. Field of the Invention

The field of this invention is the telomerase reverse transcriptase gene, specifically the regulation of the expression thereof.

2. Background of the Invention

Telomeres, which define the ends of chromosomes, consist of short, tandemly repeated DNA sequences loosely conserved in eukaryotes. Human telomeres consist of many kilobases of (TTAGGG)n together with various associated proteins. Small amounts of these terminal sequences or telomeric DNA are lost from the tips of the chromosomes during S phase because of incomplete DNA replication. Many human cells progressively lose terminal sequence with cell division, a loss that correlates with the apparent absence of telomerase in these cells. The resulting telomeric shortening has been demonstrated to limit cellular lifespan.

Telomerase is a ribonucleoprotein that synthesizes telomeric DNA. Human telomerase is made up of two components: (1) an essential structural RNA (TER) (where the human component is referred to in the art as hTER); and (2) a catalytic protein (telomerase reverse transcriptase or TERT) (where the human component is referred to in the art as hTERT). Telomerase works by recognizing the 3-prime end of DNA, e.g., telomeres, and adding multiple telomeric repeats to its 3-prime end with the catalytic protein component, e.g., hTERT, which has polymerase activity, and hTER which serves as the template for nucleotide incorporation. Of these two components of the telomerase enzyme, both the catalytic protein component and the RNA template component are activity limiting components.

Because of its role in cellular senescence and immortalization, there is much interest in the development of protocols and compositions for regulating expression of telomerase.

Relevant Literature

References of interest include WO 02/16657 and WO 02/16658 and references cited therein.

### SUMMARY OF THE INVENTION

Methods and compositions are provided for modulating, and generally upregulating, the expression of telomerase reverse transcriptase (TERT) by blocking repression of the TERT transcription, e.g., by inhibiting binding of TERT repressors to specific repressor binding sites located in the TERT promoter. Repressor binding may be blocked by addition of agents that interact with the repressor binding sites and/or the repressor proteins to prevent repression of transcription, where representative agents include double-stranded DNA reagents (i.e. “decoys”) that mimic the sequence of the repressor site, agents that bind to and block binding of repressor factors to the TERT repressor binding sites, agents that reduce the amount of TERT repressing factors available for binding to the repressor binding sites, etc. Alternatively, nucleic acid constructs are provided where the TERT repressor binding sites or portions thereof are deleted from the promoter region. Two repressor binding sites of interest, the TF-8 and TF-13 sites, are located upstream of the start of TERT coding sequence, generally in a location that is −120 to −10 relative to the start of the TERT coding sequence. In particular, the approximate locations (relative to the start of translation) of the TF-8 and TF-13 repressor binding sites are −117 to −108 and −27 to −18, respectively.

Also provided are methods of modulating the transcription repressing activity of TERT TF-8 and/or TF-13 repressor factors in order to regulate telomerase expression, which methods find use in a variety of different applications, including the immortalization of cells, the production of reagents for use in life science research, therapeutic applications and the like. In addition, methods of screening for agents that modulate TF-8 or TF-13 repression of TERT transcription are provided.

### DESCRIPTION OF THE SPECIFIC EMBODIMENTS

Nucleic acid compositions comprising deletions in the TERT minimal promoter region which include at least a partial sequence of a TERT regulatory binding site, in particular the TF-8 or TF-13 binding sites, as well as vectors and constructs including the same are provided. Also provided are methods of modulating, generally upregulating, telomerase expression by deletion or blocking of the TF-8 or TF-13 TERT repressor binding site and/or blocking repressor binding to these sites, which methods find use in a variety of different applications, including the immortalization of cells, production of reagents for use in life science research, therapeutic applications and the like. In addition, methods of screening for agents that modulate TF-8 or TF-13 repression of the TERT promoter are provided. The subject invention finds use in, among other applications, the regulation of TERT expression.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices and materials are now described.

All publications mentioned herein are incorporated herein by reference for the purpose of describing and disclosing the components that are described in the publications which might be used in connection with the presently described invention.

Nucleic Acid Compositions

As summarized above, the subject invention provides nucleic acid compositions that include a TERT promoter region comprising a deletion in the TF-8 or TF-13 TERT repressor binding sites. These specific binding sites are now described separately in greater detail.

TF-8 Repressor Binding Site

By TERT TF-8 repressor binding site is meant the site of the minimal TERT promoter that binds to a TF-8 transcription repressor protein or transcription factor, where binding of the TF-8 repressor protein to the TERT TF-8 repressor binding site results in repression of TERT expression. The subject TERT TF-8 binding site binds to a TF-8 transcription repression factor (i.e., the TERT TF-8 binding site has a sequence that is recognized by an TF-8 repressor protein).

The subject TERT TF-8 binding site is located in the region from about −117 to −106, and particularly from about −117 to −108, of the TERT promoter. The TF-8 binding site sequence is CCCTCCCAGC (SEQ ID NO:01). As such, nucleic acid compositions of this embodiment include alterations of this site, e.g., deletions or substitutions, including a deletion or substitution of all or portion of the TERT repressor binding site, e.g., preferably a deletion or substitution of at least one nucleotide, in certain embodiments at least four nucleotides within the region of nucleotides −117 to −108 (relative to the start of translation), usually at least 7 nucleotides from this region, and preferably all nucleotides from this region. Additionally, such a deletion may extend further, for example to include the nucleotides from positions −117 to −106, or subsets thereof. The subject nucleic acids of this embodiment that include a deletion (or substitution) in all or a portion of the TF-8 repressor site of the TERT promoter may be present in the genome of a cell or animal of interest, e.g., as a “knockout” deletion in a transgenic cell or animal, where the cell or animal initially has this region, or may be present in an isolated form, where basic “knockout” technology known to those skilled in the art e.g. see U.S. Pat. No. 5,464,764 to Capecchi, the disclosure of which is herein incorporated by reference. In other embodiments, the subject nucleic acids have a sequence that is substantially the same as, or identical to, the TERT TF-8 repressor binding site. A given sequence is considered to be substantially similar to this particular sequence if it shares high sequence similarity with the above described specific sequence, e.g. at least 75% sequence identity, usually at least 90%, more usually at least 95% sequence identify with the above specific sequence. Sequence similarity is calculated based on a reference sequence, which may be a subset of a larger sequence. A reference sequence will usually be at least about 6 nt long, more usually at least about 8 nt long, and may extend to the complete sequence that is being compared. Algorithms for sequence analysis are known in the art, such as BLAST, described in Altschul et al. 108(1990), J. Mol. Biol. 215:403-10

In many embodiments, the nucleic acids range in length from about 5 to 500 nt, usually from about 10 to 300 nt and more usually from about 10 to 250 nt. The nucleic acids of this embodiment include SEQ ID NO:01 or a sequence that is substantially similar or identical thereto, where this sequence may be flanked on at least one side by a domain having a sequence that is not present in a flanking domain of the wild type TERT promoter, e.g., the human TERT promoter.

Of particular interest in certain embodiments are nucleic acids of substantially the same length as the specific nucleic acid identified above, where by substantially the same length is meant that any difference in length does not exceed about 20 number %, usually does not exceed about 10 number % and 20 more usually does not exceed about 5 number %; and have sequence identity to this sequence of at least about 90%, usually at least about 95% and more usually at least about 99% over the entire length of the nucleic acid.

TF-13 Repressor Binding Site

By TERT TF-13 repressor binding site is meant the site of the minimal TERT promoter that binds to a TF-13 protein or transcription factor, where binding of the TF-13 protein to the TERT TF-13 repressor binding site results in repression of TERT expression. The subject TERT TF-13 binding site binds to a TF-13 transcription factor, i.e., the TERT TF-13 binding site has a sequence that is recognized by an TF-13 repressor protein.

The subject TERT TF-13 binding site is located in the region from about −29 to −16, and particularly from about −27 to −18, of the TERT promoter. The TF-13 binding site sequence is GAAGCCCTGG (SEQ ID NO:02). As such, nucleic acid compositions of this embodiment include alterations of this site, e.g., deletions or substitutions, including a deletion or substitution of all or portion of the TERT repressor binding site, e.g., preferably a deletion or substitution of at least one nucleotide, in certain embodiments at least four nucleotides within the region of nucleotides −27 to −18 (relative to the start of translation), usually at least 7 nucleotides from this region, and preferably all nucleotides from this region. Additionally, such a deletion may extend further, for example to include the nucleotides from positions −29 to −16, or subsets thereof. The subject nucleic acids of this embodiment that include a deletion (or substitution) in all or a portion of the TF-13 repressor site of the TERT promoter may be present in the genome of a cell or animal of interest, e.g., as a “knockout” deletion in a transgenic cell or animal, where the cell or animal initially has this region, or may be present in an isolated form. A “knockout” animal could be produced from an animal that originally has the subject TF-13 repressor site using the sequences flanking −27 to −18 described here and the basic “knockout” technology known to those skilled in the art e.g. see U.S. Pat. No. 5,464,764 to Capecchi.

In other embodiments, the subject nucleic acids have a sequence that is substantially the same as, or identical to, the TERT TF-13 repressor binding site. A given sequence is considered to be substantially similar to this particular sequence if it shares high sequence similarity with the above described specific sequence, e.g. at least 75% sequence identity, usually at least 90%, more usually at least 95% sequence identify with the above specific sequence. Sequence similarity is calculated based on a reference sequence, which may be a subset of a larger sequence. A reference sequence will usually be at least about 6 nt long, more usually at least about 8 nt long, and may extend to the complete sequence that is being compared. Algorithms for sequence analysis are known in the art, such as BLAST, described in Altschul et al. (1990), J. Mol. Biol. 215:403-10.

Of particular interest in certain embodiments are nucleic acids of substantially the same length as the specific nucleic acid identified above, where by substantially the same length is meant that any difference in length does not exceed about 20 number %, usually does not exceed about 10 number % and more usually does not exceed about 5 number %; and have sequence identity to this sequence of at least about 90%, usually at least about 95% and more usually at least about 99% over the entire length of the nucleic acid.

In many embodiments, the nucleic acids range in length from about 5 to 500 nt, usually from about 10 to 300 nt and more usually from about 10 to 250 nt. The nucleic acids of this embodiment include SEQ ID NO:02 or a sequence that is substantially similar or identical thereto, where this sequence may be flanked on at least one side by a domain having a sequence that is not present in a flanking domain of the wild type TERT promoter, e.g., the human TERT promoter.

Also provided are nucleic acids that hybridize to the above-described nucleic acids under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

In many embodiments, the above-described nucleic acid compositions include at least one repressor domain but do not include all of the components of the TERT genomic sequence, e.g., all of the other intron/exon regions of the TERT genomic sequence. In these embodiments, the subject nucleic acids include no more than about 90 number %, usually no more than about 80 number % and more usually no more than about 75 number %, where in many embodiments the subject nucleic acids include less than about 50 number %, sometimes less than about 40 number % and sometimes less than about 25 number % of the total sequence of the TERT genomic sequence. In certain embodiments, the length of the subject nucleic acids ranges from about 5 to about 5000 bases, sometimes from about 10 to about 2500 bases and usually from about 10 to about 1000 bases.

The above described nucleic acid compositions find use in a variety of different applications, including the preparation of constructs, e.g., vectors, expression systems, etc., as described more fully below, the preparation of probes for the TERT TF-8 or TF-13 repressor binding site in non-human animals, i.e., non-human homologs of the TERT TF-8 and TF-13 repressor binding sites, and the like. Where the subject nucleic acids are employed as probes, a fragment of the provided nucleic acid may be used as a hybridization probe against a genomic library from the target organism of interest, where low stringency conditions are used. The probe may be a large or small fragment, generally ranging in length from about 10 to 100 nt, usually from about 15 to 50 nt. Nucleic acids having sequence similarity are detected by hybridization under low stringency conditions, for example, at 50° C. and 6×SSC (0.9 M sodium chloride/0.09 M sodium citrate) and remain bound when subjected to washing at 55° C. in 1×SSC (0.15 M sodium chloride/0.015 M sodium citrate). Sequence identity may be determined by hybridization under stringent conditions, for example, at 50° C. or higher and 0.1×SSC (15 mM sodium chloride/01.5 mM sodium citrate). Nucleic acids having a region of substantial identity to the provided nucleic acid sequences bind to the provided sequences under stringent hybridization conditions. By using probes, particularly labeled probes of DNA sequences, one can isolate homologous or related sequences.

The subject nucleic acids are isolated and obtained in substantial purity, generally as other than an intact chromosome. As such, they are present in other than their naturally occurring environment. Usually, the DNA will be obtained substantially free of other nucleic acid sequences that do not include a TERT TF-8 or an TF-13 repressor binding site sequence or fragment thereof, generally being at least about 50%, usually at least about 90% pure and are typically “recombinant”, i.e. flanked by one or more nucleotides with which it is not normally associated on a naturally occurring chromosome.

Also provided are nucleic acid compositions that include a modified or altered repressor region, e.g., where the site includes one or more deletions or substitutions as compared to the above specific repressor regions. The subject nucleic acids of this embodiment that include a deletion (or substitution) in all or a portion of the repressor region may be present in the genome of a cell or animal of interest, e.g., as a “knockout” deletion in a transgenic cell or animal, where the cell or animal initially has this region, or may be present in an isolated form.

The subject nucleic acids may be produced using any convenient protocol, including synthetic protocols, e.g., such as those where the nucleic acid is synthesized by a sequential monomeric approach (e.g., via phosphoramidite chemistry); where subparts of the nucleic acid are so synthesize and then assembled or concatamerized into the final nucleic acid, and the like. Where the nucleic acid of interest has a sequence that occurs in nature, the nucleic acid may be retrieved, isolated, amplified et., from a natural source using conventional molecular biology protocols.

Also provided are constructs comprising the subject nucleic acid compositions, e.g., those that include either the TERT TF-8 or TF-13 repressor binding site as well as those that include a deletion in the TERT TF-8 or TF-13 repressor binding site, inserted into a vector, where such constructs may be used for a number of different applications, including propagation, screening, genome alteration, and the like, as described in greater detail below. Constructs made up of viral and non-viral vector sequences may be prepared and used, including plasmids, as desired. The choice of vector will depend on particular application in which the nucleic acid is to be employed. Certain vectors are useful for amplifying and making large amounts of the desired DNA sequence. Other vectors are suitable for expression in cells in culture, e.g., for use in screening assays. Still other vectors are suitable for transfer and expression in cells in a whole animal or person. The choice of appropriate vector is well within the skill of the art. Many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example. Additional examples of nucleic acid compositions that include either the TERT TF-8 or the TF-13 repressor binding site are polymers, e.g. a double stranded DNA molecules, that mimic the TF-8 or TF-13 TERT repressor sites as described above. Nucleic acid compositions of further interest are anti-sense sequences which are sufficiently homologous to the TERT TF-8 or the TERT TF-13 binding site,

Also provided are expression cassettes, vectors or systems that find use in, among other applications, screening for agents that modulate, e.g., inhibit or enhance the repressive activity of the region, as described in greater detail below; and/or to provide for expression of proteins under the control of the expression regulation mechanism of the TERT gene. By expression cassette or system is meant a nucleic acid that includes a sequence encoding a peptide or protein of interest, i.e., a coding sequence, operably linked to a promoter sequence, where by operably linked is meant that expression of the coding sequence is under the control of the promoter sequence. The expression systems and cassettes of the subject invention comprise a TERT TF-8 and/or TF-13 repressor binding site/region operably linked to the promoter, where the promoter is, in many embodiments, a TERT promoter, such as the hTERT promoter. See e.g., the hTERT promoter sequence described in Cong et al., Hum. Mol. Genet. (1999) 8:137-142.

As indicated above, expression systems comprising the subject regions find use in applications where it is desired to control expression of a particular coding sequence using the TERT transcriptional mechanism. In such applications, the expression system further includes the coding sequence of interest operably linked to the TERT promoter/TERT repressor binding site elements of interest. The expression system is then employed in an appropriate environment to provide expression or non-expression of the protein, as desired, e.g., in an environment in which telomerase is expressed, e.g., a Hela cell, or in an environment in which telomerase is not expressed, e.g., an MRC5 cell. Alternatively, the expression system may be used in an environment in which telomerase expression is inducible, e.g., by adding to the system an additional agent that turns on telomerase expression.

The above applications of the subject nucleic acid compositions are merely representative of the diverse applications in which the subject nucleic acid compositions find use.

Methods of Enhancing Tert Expression

Also provided are methods of modulating, and generally enhancing, TERT expression. Specifically, methods are provided for enhancing TERT expression utilizing an expression system that includes an operably linked TERT coding sequence, a TERT promoter and a TERT TF-8 or TERT TF-13 repressor binding site, e.g. a TERT expression system such as is found in the hTERT genomic sequence. By enhancing is meant that the expression level of the TERT coding sequence is increased by at least about 2 fold, usually by at least about 5 fold and sometimes by at least 25, 50, 100 fold and in particular about 300 fold or higher, as compared to a control, i.e., expression from an expression system that is not subjected to the methods of the present invention. Alternatively, in cases where expression of the TERT gene is so low that it is undetectable, expression of the TERT gene is considered to be enhanced if expression is increased to a level that is easily detectable.

In these methods, either TF-13 or TF-8 repression of TERT expression is inhibited. By inhibited is meant that the repressive activity of the TERT TF-8 repressor binding site/TF-8 repressor interaction or the TERT TF-13 repressor binding site/TF-13 repressor interaction is decreased with respect to TERT expression by a factor sufficient to provide for the desired enhanced level of TERT expression, as described above, e.g. by at least about 1.2-2 fold, usually by at least about 5 fold. Inhibition of the repression of TERT transcription by a TF-8 or TF-13 site repressor may be accomplished in a number of ways. Representative protocols for inhibiting TF-13 and TF-8 repression are now provided.

One representative method of inhibiting repression of transcription is to employ double-stranded, i.e., duplex, oligonucleotide decoys specific for proteins that bind to either the TERT TF-8 and/or TF-13 repressor binding site which causes transcription repression. These duplex oligonucleotide decoys will have at least the sequence of a TERT repressor binding site (TF-8 or TF-13) that is required to bind to the respective target protein. In many embodiments, the length of these duplex oligonucleotide decoys ranges from about 5 to 5000, usually from about 5 to 500 and more usually from about 10 to 50 bases. In using such oligonucleotide decoys, the decoys are placed into the environment of the expression system and the target protein, resulting in de-repression of the transcription and expression of the TERT coding sequence. Oligonucleotide decoys and methods for their use and administration are further described in general terms in Morishita et al., Circ Res (1998) 82 (10):1023-8. These oligonucleotide decoys generally include a TERT repressor binding site recognized by either the TF-8 or TF-13 target protein, including the specific regions detailed above, where these particular embodiments are nucleic acid compositions of the subject invention, as defined above.

Instead of the above described decoys, other agents that disrupt binding of target proteins to the subject TERT TF-8 or TF-13 repressor binding sites and thereby inhibit repression may be employed. Other agents of interest include, among other types of agents, small molecules that bind to either TF-8 or TF-13 and inhibit their binding to their specific TERT repressor region. Naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below. Small molecule agents of particular interest include pyrrole-imidazole polyamides, analogous to those described in Dickinson et al., Biochemistry 1999 Aug. 17; 38 (33): 10801-7.

Alternatively, agents that disrupt protein-protein interactions of the transacting factor with cofactors, e.g., cofactor binding, and thereby inhibit the transacting factor's binding to the target repressor site/region are of interest.

In another embodiment, the TF-8 or TF-13 repressor binding site is inactivated so that it no longer represses transcription. By inactivated is meant that the repressor binding site of interest is genetically modified so that it no longer represses TERT transcription and expression. One means of inactivating the TERT TF-8 repressor binding site is to alter or mutate it so that it is no longer capable of repression, e.g., so that it is no longer bound by the TF-8 repressor protein that binds to it and cause transcription repression. In a similar manner, inactivating the TERT TF-13 repressor binding site means to alter or mutate the site so that it is no longer capable of repression, e.g., so that it is no longer bound by the TF-13 protein that binds to it and cause transcription repression. The alteration or mutation may take a number of different forms, e.g., through deletion of one or more nucleotide residues in the repressor region, through exchange of one or more nucleotide residues in the repressor region, and the like. One means of making such alterations in the repressor region of the target expression system is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

In yet other embodiments, expression of regulatory proteins or factors that bind to either the TERT TF-8 or TF-13 repressor binding site to inhibit TERT transcription and expression are inhibited. Inhibition of target TF-13 factor expression may be accomplished using any convenient means, including administration of an agent, that inhibits TF-13 expression, inactivation of the target TF-13 gene, e.g., through recombinant techniques, etc. Inhibiting the expression of target TF-8 repressor factor maybe accomplished by administering an agent that inhibits TF-8 repressor factor expression, inactivation of the target TF-8 repressor factor gene, e.g., through recombinant techniques, etc., or any other convenient means.

The above-described methods of enhancing TERT expression find use in a number of different applications. In many applications, the subject methods and compositions are employed to enhance TERT expression in a cell that endogenously comprises a TERT gene, e.g. for enhancing expression of hTERT in a normal human cell in which TERT expression is repressed. The target cell of these applications is, in many instances, a normal cell, e.g. a somatic cell. Expression of the TERT gene is considered to be enhanced if, consistent with the above description, expression is increased by at least about 2 fold, usually at least about 5 fold and often 25, 50, 100 fold or higher, as compared to a control, e.g., an otherwise identical cell not subjected to the subject methods. Alternatively, in cases where expression of the TERT gene is so low that it is undetectable, expression of the TERT gene is considered to be enhanced if expression is increased to a level that is easily detectable.

A more specific application in which the subject methods find use is to increase the proliferative capacity of a cell. The term “proliferative capacity” as used herein refers to the number of divisions that a cell can undergo, and preferably to the ability of the target cell to continue to divide where the daughter cells of such divisions are not transformed, i.e., they maintain normal response to growth and cell cycle regulation. The subject methods typically result in an increase in proliferative capacity of at least about 1.2-2 fold, usually at least about 5 fold and often at least about 10, 20, 50 fold or even higher, compared to a control. As such, yet another more specific application in which the subject methods find use is in the delay of the occurrence of cellular senescence. By practicing the subject methods, the onset of cellular senescence may be delayed by a factor of at least about 1.2-2 fold, usually at least about 5 fold and often at least about 10, 20, 50 fold or even higher, compared to a control.

Generation of Antibodies

In one embodiment of the invention, the blocking of the TF-8 or TF-13 TERT repressor site and/or repressors specific for either of these sites, is used to immortalize cells in culture. Exemplary of cells that may be used for this purpose are non-transformed antibody producing cells, e.g. B cells and plasma cells which may be isolated and identified for their ability to produce a desired antibody using known technology as, for example, taught in U.S. Pat. No. 5,627,052. These cells may either secrete antibodies (antibody-secreting cells) or maintain antibodies on the surface of the cell without secretion into the cellular environment. Such cells have a limited lifespan in culture, and are usefully immortalized by upregulating expression of telomerase using the methods of the present invention.

Because the above-described methods are methods of increasing expression of TERT and therefore increasing the proliferative capacity and/or delaying the onset of senescence in a cell, they find applications in the production of a range of reagents, typically cellular or animal reagents. For example, the subject methods may be employed to increase proliferation, delay senescence and/or extend the lifetimes of cultured cells. Cultured cell populations having enhanced TERT expression are produced using any of the protocols as described above, including by contact with an agent that inhibits repressor region transcription repression and/or modification of the repressor region in a manner such that it no longer represses TERT coding sequence transcription, etc.

Therapeutic Applications

The methods also find use in a variety of therapeutic applications in which it is desired to increase or enhance TERT expression in a target cell or collection of cells, where the collection of cells may be a whole animal or portion thereof, e.g., tissue, organ, etc. As such, the target cell(s) may be a host animal or portion thereof, or may be a therapeutic cell (or cells) which is to be introduced into a multicellular organism, e.g., a cell employed in gene therapy. In such methods, an effective amount of an active agent that inhibits TF-8 or TF-13 repression of TERT transcription is administered to the target cell or cells, e.g., by contacting the cells with the agent, by administering the agent to the animal, etc. By effective amount is meant a dosage sufficient to enhance TERT expression in the target cell(s), as described above.

In the subject methods, the active agent(s) may be administered to the targeted cells using any convenient means capable of resulting in the desired enhancement of TERT expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. oligonucleotide decoy, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. For nucleic acid therapeutic agent, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

The subject methods find use in the treatment of a variety of different conditions in which the enhancement of TERT expression in the host is desired. By treatment is meant that at least an amelioration of the symptoms associated with the condition afflicting the host is achieved, where amelioration is used in a broad sense to refer to at least a reduction in the magnitude of a parameter, e.g. symptom (such as inflammation), associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

One representative disease condition that may be treated according to the subject invention is Progeria, or Hutchinson-Gilford syndrome. This condition is a disease of shortened telomeres for which no known cure exists. It afflicts children, who seldom live past their early twenties. In many ways progeria parallels aging itself. However, these children are born with short telomeres. Their telomeres don't shorten at a faster rate; they are just short to begin with. The subject methods can be used in such conditions to further delay natural telomeric shortening and/or increase telomeric length, thereby treating this condition.

Another specific disease condition in which the subject methods find use is in immune senescence. The effectiveness of the immune system decreases with age. Part of this decline is due to fewer T-lymphocytes in the system, a result of lost replicative capacity. Many of the remaining T-lymphocytes experience loss of function as their telomeres shorten and they approach senescence. The subject methods can be employed to inhibit immune senescence due to telomere loss. Because hosts with aging immune systems are at greater risk of developing pneumonia, cellulitis, influenza, and many other infections, the subject methods reduce morbidity and mortality due to infections.

The subject methods also find use in AIDS therapy. HIV, the virus that causes AIDS, invades white blood cells, particularly CD4 lymphocyte cells, and causes them to reproduce high numbers of the HIV virus, ultimately killing cells. In response to the loss of immune cells (typically about a billion per day), the body produces more CD8 cells to be able to suppress infection. This rapid cell division accelerates telomere shortening, ultimately hastening immune senescence of the CD8 cells. Anti-retroviral therapies have successfully restored the immune systems of AIDS patients, but survival depends upon the remaining fraction of the patient's aged T-cells. Once shortened, telomere length has not been naturally restored within cells. The subject methods can be employed to restore this length and/or prevent further shortening. As such the subject methods can spare telomeres and is useful in conjunction with the anti-retroviral treatments currently available for HIV.

Yet another type of disease condition in which the subject methods find use is cardiovascular disease. The subject methods can be employed to extend telomere length and replicative capacity of endothelial cells lining of blood vessel walls (DeBono, Heart 80:110-1, 1998). Endothelial cells form the inner lining of blood vessels and divide and replace themselves in response to stress. Stresses include high blood pressure, excess cholesterol, inflammation, and flow stresses at forks in vessels. As endothelial cells age and can no longer divide sufficiently to replace lost cells, areas under the endothelial layer become exposed. Exposure of the underlying vessel wall increases inflammation, the growth of smooth muscle cells, and the deposition of cholesterol. As a result, the vessel narrows and becomes scarred and irregular, which contributes to even more stress on the vessel (Cooper, Cooke and Dzau, J Gerontol Biol Sci 49: 191-6, 1994). Aging endothelial cells also produce altered amounts of trophic factors (hormones that affect the activity of neighboring cells). These too contribute to increased clotting, proliferation of smooth muscle cells, invasion by white blood cells, accumulation of cholesterol, and other changes, many of which lead to plaque formation and clinical cardiovascular disease (Ibid.). By extending endothelial cell telomeres, the subject methods can be employed to combat the stresses contributing to vessel disease. Many heart attacks may be prevented if endothelial cells were enabled to continue to divide normally and better maintain cardiac vessels. The occurrence of strokes caused by the aging of brain blood vessels may also be significantly reduced by employing the subject methods to help endothelial cells in the brain blood vessels to continue to divide and perform their intended function.

The subject methods also find use in skin rejuvenation. The skin is the first line of defense of the immune system and shows the most visible signs of aging (West, Arch Dermatol 130(1):87-95, 1994). As skin ages, it thins, develops wrinkles, discolors, and heals poorly. Skin cells divide quickly in response to stress and trauma; but, over time, there are fewer and fewer actively dividing skin cells. Compounding the loss of replicative capacity in aging skin is a corresponding loss of support tissues. The number of blood vessels in the skin decreases with age, reducing the nutrients that reach the skin. Also, aged immune cells less effectively fight infection. Nerve cells have fewer branches, slowing the response to pain and increasing the chance of trauma. In aged skin, there are also fewer fat cells, increasing susceptibility to cold and temperature changes. Old skin cells respond more slowly and less accurately to external signals. They produce less vitamin D, collagen, and elastin, allowing the extracellular matrix to deteriorate. As skin thins and loses pigment with age, more ultraviolet light penetrates and damages skin. To repair the increasing ultraviolet damage, skin cells need to divide to replace damaged cells, but aged skin cells have shorter telomeres and are less capable of dividing (Fossel, REVERSING HUMAN AGING. William Morrow & Company, New York City, 1996).

By practicing the subject methods, e.g., via administration of an active agent topically, one can extend telomere length, and slow the downward spiral that skin experiences with age. Such a product not only helps protect a person against the impairments of aging skin; it also permits rejuvenated skin cells to restore youthful immune resistance and appearance. The subject methods can be used for both medical and cosmetic skin rejuvenation applications.

Yet another disease condition in which the subject methods find use in the treatment of osteoporosis. Two types of cells interplay in osteoporosis: osteoblasts make bone and osteoclasts destroy it. Normally, the two are in balance and maintain a constant turnover of highly structured bone. In youth, bones are resilient, harder to break, and heal quickly. In old age, bones are brittle break easily, and heal slowly and often improperly. Bone loss has been postulated to occur because aged osteoblasts, having lost much of their replicative capacity, cannot continue to divide at the rate necessary to maintain balance (Hazzard et al. PRINCIPLES OF GERIATRIC MEDICINE AND GERONTOLOGY, 2d ed. McGraw-Hill, New York City, 1994). The subject methods can be employed to lengthen telomeres of osteoblast and osteoclast stem cells, thereby encouraging bone replacement and proper remodeling and reinforcement. The resultant stronger bone improves the quality of life for the many sufferers of osteoporosis and provides savings from fewer fracture treatments. The subject methods are generally part of a comprehensive treatment regime that also includes calcium, estrogen, and exercise.

Additional disease conditions in which the subject methods find use are described in WO 99/35243, the disclosures of which are herein incorporated by reference.

In addition to the above described methods, the subject methods can also be used to extend the lifetime of a mammal. By extend the lifetime is meant to increase the time during which the animal is alive, where the increase is generally at least 1%, usually at least 5% and more usually at least about 10%, as compared to a control.

As indicated above, instead of a multicellular animal, the target may be a cell or population of cells which are treated according to the subject methods and then introduced into a multicellular organism for therapeutic effect. For example, the subject methods may be employed in bone marrow transplants for the treatment of cancer and skin grafts for burn victims. In these cases, cells are isolated from a human donor and then cultured for transplantation back into human recipients. During the cell culturing, the cells normally age and senesce, decreasing their useful lifespans. Bone marrow cells, for instance, lose approximately 40% of their replicative capacity during culturing. This problem is aggravated when the cells are first genetically engineered (Decary, Mouly et al. Hum Gene Ther 7(11): 1347-50, 1996). In such cases, the therapeutic cells must be expanded from a single engineered cell. By the time there are sufficient cells for transplantation, the cells have undergone the equivalent of 50 years of aging (Decary, Mouly et al. Hum Gene Ther 8(12): 1429-38, 1997). Use of the subject methods spares the replicative capacity of bone marrow cells and skin cells during culturing and expansion and thus significantly improves the survival and effectiveness of bone marrow and skin cell transplants. Any transplantation technology requiring cell culturing can benefit from the subject methods, including ex vivo gene therapy applications in which cells are cultured outside of the animal and then administered to the animal, as described in U.S. Pat. Nos. 6,068,837; 6,027,488; 5,824,655; 5,821,235; 5,770,580; 5,756,283; 5,665,350; the disclosures of which are herein incorporated by reference.

Screening Assays

Also provided by the subject invention are screening protocols and assays for identifying agents that modulate, e.g., inhibit or enhance, TF-8 or TF-13 repression of TERT transcription. The screening methods will typically be assays which provide for qualitative/quantitative measurements of TERT promoter controlled expression, e.g. of a coding sequence for a marker or reporter gene, in the presence of a particular candidate therapeutic agent. For example, the assay could be an assay which measures the TERT promoter controlled expression of a reporter gene (i.e. coding sequence, e.g., luciferase, SEAP, etc.) in the presence and absence of a candidate inhibitor agent, e.g. the expression of the reporter gene in the presence or absence of a candidate agent. The screening method may be an in vitro or in vivo format, where both formats are readily developed by those of skill in the art. Whether the format is in vivo or in vitro, an expression system (e.g., a plasmid) that includes a repressor binding site of interest, a TERT promoter and a reporter coding sequence all operably linked, is combined with the candidate agent in an environment in which, in the absence of the candidate agent, the TERT promoter is repressed, e.g., in the presence of a repressor protein that interacts with the TERT TF-8 or TF-13 binding site and causes TERT promoter repression. The conditions may be set up in vitro by combining the various required components in an aqueous medium, or the assay may be carried out in vivo, e.g., in a cell that normally lacks telomerase activity, e.g., an MRC5 cell, etc.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents identified in the above screening assays that inhibit TF-8 or TF-13 repression of TERT transcription find use in the methods described above, e.g., in the enhancement of TERT expression.

Alternatively, agents identified in the above screening assays that enhance TF-8 or TF-13 repression find use in applications where inhibition of TERT expression is desired, e.g., in the treatment of disease conditions characterized by the presence of unwanted TERT expression, such as cancer and other diseases characterized by the presence of unwanted cellular proliferation, where such methods are described in, for example, U.S. Pat. Nos. 5,645,986; 5,656,638; 5,703,116; 5,760,062; 5,767,278; 5,770,613; and 5,863,936; the disclosures of which are herein incorporated by reference.

Also provided are methods of treating disease conditions characterized by the presence of unwanted or undesired TERT expression. In such methods, TERT expression is suppressed using the subject methods to effect the desired treatment. Such disease conditions include cancer/neoplastic diseases and other diseases characterized by the presence of unwanted cellular proliferation, e.g., hyperplasias, where such methods are described in, for example, U.S. Pat. Nos. 5,645,986; 5,656,638; 5,703,116; 5,760,062; 5,767,278; 5,770,613; and 5,863,936; the disclosures of which are herein incorporated by reference. As such, the methods of the present invention can provide a highly general method of treating many-if not most-malignancies, as demonstrated by the highly varied human tumor cell lines and tumors having telomerase activity, including tumors derived from cells selected from skin, connective tissue, adipose, breast, lung, stomach, pancreas, ovary, cervix, uterus, kidney, bladder, colon, prostate, central nervous system (CNS), retina and blood, and the like. More importantly, the subject methods can be effective in providing treatments that discriminate between malignant and normal cells to a high degree, avoiding many of the deleterious side-effects present with most current chemotherapeutic regimes which rely on agents that kill dividing cells indiscriminately.

Antibody Production

The subject methods find use in the generation of monoclonal antibodies. An antibody-forming cell may be identified among antibody-forming cells obtained from an animal which has either been immunized with a selected substance, or which has developed an immune response to an antigen as a result of disease. Animals may be immunized with a selected antigen using any of the techniques well known in the art suitable for generating an immune response. Antigens may include any substance to which an antibody may be made, including, among others, proteins, carbohydrates, inorganic or organic molecules, and transition state analogs that resemble intermediates in an enzymatic process. Suitable antigens include, among others, biologically active proteins, hormones, cytokines, and their cell surface receptors, bacterial or parasitic cell membrane or purified components thereof, and vital antigens.

As will be appreciated by one of ordinary skill in the art, antigens which are of low immunogenicity may be accompanied with an adjuvant or hapten in order to increase the immune response (for example, complete or incomplete Freund's adjuvant) or with a carrier such as keyhole limpet hemocyanin (KLH).

Procedures for immunizing animals are well known in the art. Briefly, animals are injected with the selected antigen against which it is desired to raise antibodies. The selected antigen may be accompanied by an adjuvant or hapten, as discussed above, in order to further increase the immune response. Usually the substance is injected into the peritoneal cavity, beneath the skin, or into the muscles or bloodstream. The injection is repeated at varying intervals and the immune response is usually monitored by detecting antibodies in the serum using an appropriate assay that detects the properties of the desired antibody. Large numbers of antibody-forming cells can be found in the spleen and lymph node of the immunized animal. Thus, once an immune response has been generated, the animal is sacrificed, the spleen and lymph nodes are removed, and a single cell suspension is prepared using techniques well known in the art.

Antibody-forming cells may also be obtained from a subject which has generated the cells during the course of a selected disease. For instance, antibody-forming cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-forming cells may be obtained from subjects with disease due to known etiological agents such as malaria or AIDS. These antibody forming cells may be derived from the blood or lymph nodes, as well as from other diseased or normal tissues. Antibody-forming cells may be prepared from blood collected with an anticoagulant such as heparin or EDTA. The antibody-forming cells may be further separated from erythrocytes and polymorphs using standard procedures such as centrifugation with Ficoll-Hypaque (Pharmacia, Uppsula, Sweden). Antibody-forming cells may also be prepared from solid tissues such as lymph nodes or tumors by dissociation with enzymes such as collagenase and trypsin in the presence of EDTA.

Antibody-forming cells may also be obtained by culture techniques such as in vitro immunization. Briefly, a source of antibody-forming cells, such as a suspension of spleen or lymph node cells, or peripheral blood mononuclear cells are cultured in medium such as RPMI 1640 with 10% fetal bovine serum and a source of the substance against which it is desired to develop antibodies. This medium may be additionally supplemented with amounts of substances known to enhance antibody-forming cell activation and proliferation such as lipopolysaccharide or its derivatives or other bacterial adjuvants or cytokines such as IL-1, IL-2, IL-4, IL-5, IL-6, GM-CSF, and IFN-.gamma. To enhance immunogenicity, the selected antigen may be coupled to the surface of cells, for example, spleen cells, by conventional techniques such as the use of biotin/avidin as described below.

Antibody-forming cells may also be obtained from very early monoclonal or oligoclonal fusion cultures produced by conventional hybridoma technology. The present invention is advantageous in that it allows rapid selection of antibody-forming cells from unstable, interspecies hybridomas, e.g., formed by fusing antibody-forming cells from animals such as rabbits, humans, cows, pigs, cats, and dogs with a murine myeloma such NS-1.

Antibody-forming cells may be enriched by methods based upon the size or density of the antibody-forming cells relative to other cells. Gradients of varying density of solutions of bovine serum albumin can also be used to separate cells according to density. The fraction that is most enriched for desired antibody-forming cells can be determined in a preliminary procedure using the appropriate indicator system in order to establish the antibody-forming cells.

The identification and culture of antibody producing cells of interest is followed by enhancement of TERT expression is these cells by the subject methods, thereby avoiding the need for the immortalization/fusing step employed in traditional hybridoma manufacture protocols. In such methods, the first step is immunization of the host animal with an immunogen, typically a polypeptide, where the polypeptide will preferably be in substantially pure form, comprising less than about 1% contaminant. The immunogen may comprise the complete protein, fragments or derivatives thereof. To increase the immune response of the host animal, the protein may be combined with an adjuvant, where suitable adjuvants include alum, dextran, sulfate, large polymeric anions, oil & water emulsions, e.g. Freund's adjuvant, Freund's complete adjuvant, and the like. The protein may also be conjugated to synthetic carrier proteins or synthetic antigens. A variety of hosts may be immunized to produce the subject antibodies. Such hosts include rabbits, guinea pigs, rodents, e.g. mice, rats, sheep, goats, and the like. The protein is administered to the host, usually intradermally, with an initial dosage followed by one or more, usually at least two, additional booster dosages. Following immunization, generally, the spleen and/or lymph nodes of an immunized host animal provide a source of plasma cells. The plasma cells are treated according to the subject invention to enhance TERT expression and thereby, increase the proliferative capacity and/or delay senescence to produce “pseudo” immortalized cells. Culture supernatant from individual cells is then screened using standard techniques to identify those producing antibodies with the desired specificity. Suitable animals for production of monoclonal antibodies to a human protein include mouse, rat, hamster, etc. To raise antibodies against the mouse protein, the animal will generally be a hamster, guinea pig, rabbit, etc. The antibody may be purified from the cell supernatants or ascites fluid by conventional techniques, e.g. affinity chromatography using RFLAT-1 protein bound to an insoluble support, protein A sepharose, etc.

In an analogous fashion, the subject methods are employed to enhance TERT expression in non-human animals, e.g., non-human animals employed in laboratory research. Using the subject methods with such animals can provide a number of advantages, including extending the lifetime of difficult and/or expensive to produce transgenic animals. As with the above described cells and cultures thereof, the expression of TERT in the target animals may be enhanced using a number of different protocols, including the administration of an agent that inhibits TF-8 repression and/or targeted disruption of the TF-8 repressor binding site. In a similar manner, the expression of TERT in target animals as well as the cells and cultures described above, may include the administration of an agent that inhibits TF-13 repression and/or targeted disruption of the TF-13 repressor binding site. The subject methods may be used with a number of different types of animals, where animals of particular interest include mammals, e.g., rodents such as mice and rats, cats, dogs, sheep, rabbits, pigs, cows, horses, and non-human primates, e.g. monkeys, baboons, etc.

The following examples are offered by way of illustration and not by way of limitation.

### Experimental

TERT Minimal Promoter Deletion Experiments

118 deletions of the minimal telomerase promoter were constructed and assayed for their affects on the TERT minimal promoter's ability to drive the expression of the SEAP reporter gene in transient transfection assays. The sequence of the minimal promoter is shown in Table 1. Table 1 shows the “A” of the telomerase translation initiation codon “ATG” as base number “1”. Also shown in Table 1 is the sequence AATTCGCCCACC (SEQ ID NO:03) which was inserted immediately upstream of the “ATG codon to optimize translation and to provide a convenient restriction site (ECOR1) to simplify constructions. As seen, these additional bases are not included in the numbering scheme used to identify bases in the minimal promoter. Bases upstream of base −258 are sequences from the vector used in the construction.

TABLE 1

| | |
|---|---|
| −258 −250 −240 −230 | (SEQ ID NO:04) |
| \| \| \| \| | |
| CGCGTGCTAG CCCGGGCTCG AGCCAGGACC GCGCTCCCCA CGTGGCGGAG GGACTGGGGA | |
| −220 −210 −200 −190 −180 −170 | |
| \| \| \| \| \| \| | |
| CCCGGGCACC CGTCCTGCCC CTTCACCTTC CAGCTCCGCC TCCTCCGCGC GGACCCCGCC | |
| −160 −150 −140 −130 −120 −110 | |
| \| \| \| \| \| \| | |
| CCGTCCCGAC CCCTCCCGGG TCCCCGGCCC AGCCCCCTCC GGGCCCTCCC AGCCCCTCCC | |
| −100 −90 −80 −70 −60 −50 | |
| \| \| \| \| \| \| | |
| CTTCCTTTCC GCGGCCCCGC CCTCTCCTCG CGGCGCGAGT TTCAGGCAGC GCTGCGTCCT | |
| −40 −30 −20 −10 −1 +1 | |
| \| \| \| \| \| \| | |
| GCTGCGCACG TGGGAAGCCC TGGCCCCGGC CACCCCCGCG AATTCGCCCA CCATG | |

The 118 individual deletions made of the minimal telomerase promoter sequence are listed below in Table 2:

TABLE 2

| # | Deletion |
|---|---|
| 1 | −258 to −248 |
| 2 | −258 to −228 |
| 3 | −258 to −208 |
| 4 | −258 to −188 |
| 5 | −258 to −168 |
| 6 | −258 to −148 |
| 7 | −258 to −128 |
| 8 | −258 to −108 |
| 9 | −258 to −88 |
| 10 | −258 to −68 |
| 11 | −258 to −48 |
| 12 | −258 to −28 |
| 13 | −258 to −8 |
| 14 | −258 to −1 |
| 15 | −257 to −248 |
| 16 | −257 to −228 |
| 17 | −257 to −208 |
| 18 | −257 to −188 |
| 19 | −257 to −168 |
| 20 | −257 to −148 |
| 21 | −257 to −128 |
| 22 | −257 to −108 |
| 23 | −257 to −88 |
| 24 | −257 to −68 |
| 25 | −257 to −48 |
| 26 | −257 to −28 |
| 27 | −257 to −8 |
| 28 | −257 to −1 |
| 29 | −237 to −228 |
| 30 | −237 to −208 |
| 31 | −237 to −188 |
| 32 | −237 to −168 |
| 33 | −237 to −148 |
| 34 | −237 to −128 |
| 35 | −237 to −108 |
| 36 | −237 to −88 |
| 37 | −237 to −68 |
| 38 | −237 to −48 |
| 39 | −237 to −28 |
| 40 | −237 to −8 |
| 41 | −237 to −1 |
| 42 | −217 to −208 |
| 43 | −217 to −188 |
| 44 | −217 to −168 |
| 45 | −217 to −148 |
| 46 | −217 to −128 |
| 47 | −217 to −108 |
| 48 | −217 to −88 |
| 49 | −217 to −68 |
| 50 | −217 to −48 |
| 51 | −217 to −28 |
| 52 | −217 to −8 |
| 53 | −217 to −1 |
| 54 | −197 to −188 |
| 55 | −197 to −168 |
| 56 | −197 to −148 |
| 57 | −197 to −128 |
| 58 | −197 to −108 |
| 59 | −197 to −88 |
| 60 | −197 to −68 |
| 61 | −197 to −48 |
| 62 | −197 to −28 |
| 63 | −197 to −8 |
| 64 | −197 to −1 |
| 65 | −177 to −168 |
| 66 | −177 to −148 |
| 67 | −177 to −128 |
| 68 | −177 to −108 |
| 69 | −177 to −88 |
| 70 | −177 to −68 |
| 71 | −177 to −48 |
| 72 | −177 to −28 |
| 73 | −177 to −8 |
| 74 | −177 to −1 |
| 75 | −157 to −148 |
| 76 | −157 to −128 |
| 77 | −157 to −108 |
| 78 | −157 to −88 |
| 79 | −157 to −68 |
| 80 | −157 to −48 |
| 81 | −157 to −28 |
| 82 | −157 to −8 |
| 83 | −157 to −1 |
| 84 | −137 to −128 |
| 85 | −137 to −108 |
| 86 | −137 to −88 |
| 87 | −137 to −68 |
| 88 | −137 to −48 |
| 89 | −137 to −28 |
| 90 | −137 to −8 |
| 91 | −137 to −1 |
| 92 | −117 to −108 |
| 93 | −117 to −88 |
| 94 | −117 to −68 |
| 95 | −117 to −48 |
| 96 | −117 to −28 |
| 97 | −117 to −8 |
| 98 | −117 to −1 |
| 99 | −97 to −88 |
| 100 | −97 to −68 |
| 101 | −97 to −48 |
| 102 | −97 to −28 |
| 103 | −97 to −8 |
| 104 | −97 to −1 |
| 105 | −77 to −68 |
| 106 | −77 to −48 |
| 107 | −77 to −28 |
| 108 | −77 to −8 |
| 109 | −77 to −1 |
| 110 | −57 to −48 |
| 111 | −57 to −28 |
| 112 | −57 to −8 |
| 113 | −57 to −1 |
| 114 | −37 to −28 |
| 115 | −37 to −8 |
| 116 | −37 to −1 |
| 117 | −17 to −8 |
| 118 | −17 to −1 |

In all cases, the deleted region was replaced with a HinDIII site to allow easy verification of each deletion.

Analysis of each of the 118 deletions in a transient transfection expression assay (utilizing SEAP as an expression reporter gene) resulted in the identification of several repressor transcription factor sites that control the regulation of the minimal telomerase promoter. Two of these repressor transcription factor (TF) sites are shown below in Table 3:

TABLE 3

| | | Centered | | | |
|---|---|---|---|---|---|
| TF | Transcription | Around | Identified | Identified | |
| Number | Factor Site | Base # | in HELA | in MRC5 | Comments |
| 8 | Repressor | −112 | Weak | Yes | |
| 13 | Repressor | −22 | Yes | no | |

Table 4 shows the DNA sequence of the telomerase minimal promoter. Table 4 includes the published potential transcription factor sites above the sequence and the TF-8 and TF-13 repressor transcription factor sites found using the minimal promoter deletions are indicated below the sequence.

The overlap between the published sites and the TF-8 site suggest that TF-8 may be a possible SP1 binding site and/or site for other transcription factors that bind GC-Boxes. It should also be noted that there is no correlation between the TF-8 or TF-13 sites and the published E-boxes (sites recognized by the Myc family of transcription factors as well as USF).

TABLE 4

| | | |
|---|---|---|
| | −258 E-Box | (SEQ ID NO:05) |
| \| ****** | | |
| | CGCGTGCTAGCCCGGGCTCGAGCCAGGACCGCGCTCCCCACGTGGCGGAGGGACTGGGGA | |
| | SP1 SP1 | |
| | ********* ******* | |
| | GC-Box GC-Box | |
| | ************** ********** | |
| −220 | CCCGGGCACCCGTCCTGCCCCTTCACCTTCCAGCTCCGCCTCCTCCGCGCGGACCCCGCC | |
| | SP1 SP1 | |
| | ** ********* ********* | |
| | GC-Box GC-Box | |
| | **** ************** *********** | |
| −160 | CCGTCCCGACCCCTCCCGGGTCCCCGGCCCAGCCCCCTCCGGGCCCTCCCAGCCCCTCCC | |
| | TF-8 | |
| | Repressor | |
| | SP1 | |
| | ********* | |
| | GC-Box | |
| | *** ************** | |
| −100 | CTTCCTTTCCGCGGCCCCGCCCTCTCCTCGCGGCGCGAGTTTCAGGCAGCGCTGCGTCCT | |
| | E-Box | |
| | ****** | |
| −40 | GCTGCGCACGTGGGAAGCCCTGGCCCCGGCCACCCCCGCGAATTCGCCCACCATG | |
| | TF-13 | |

These examples indicate that the inactivation of transcription factors that bind to TF-13 and/or TF-8 should be an effective way to increase telomerase expression enough to maintain and/or increase the length of telomeres in normal cells. This study also indicates that telomerase expression can be decreased (e.g. for cancer treatment) by controlling the regulation of expression (or activity) of the transcription factors that bind TF-13 and/or TF-8 in cells that express telomerase.

It is evident from the above results and discussion that the subject invention provides important new nucleic acid compositions that find use in a variety of applications, including the establishment of expression systems that exploit the regulatory mechanism of the TERT gene and the establishment of screening assays for agents that enhance TERT expression. In addition, the subject invention provides methods of enhancing TERT expression in a cellular or animal host, which methods find use in a variety of applications, including the production of scientific research reagents and therapeutic treatment applications. Accordingly, the subject invention represents significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A nucleic acid present in other than its natural environment, wherein said nucleic acid has a nucleotide sequence that is the same as or substantially identical to the TERT TF-8 and/or TF-13 repressor binding site.

2. The nucleic acid according to claim 1, wherein said nucleic acid has a length ranging from about 1 to 50 bases.

3. The nucleic acid according to claim 1, wherein said nucleic acid is isolated

4. The nucleic acid according to claim 1, wherein said nucleic acid has a sequence that is substantially the same as or identical to a sequence found in SEQ ID NO:01.

5. An isolated nucleic acid or mimetic thereof that hybridizes under stringent conditions to the nucleic acid according to claims 1 to 4 or its complementary sequence.

6. A construct comprising a nucleic acid according to claims 1 to 5.

7. The construct according to claim 6, wherein said construct comprises a TERT promoter.

8. The construct according to claim 6, wherein said construct is an expression cassette.

9. A method of enhancing expression of TERT from a TERT expression system that includes a TERT promoter and a TERT TF-8 and/or TF-13 repressor binding site, said method comprising:
inhibiting TERT transcription repression by said TERT TF-8 or TF-13 repressor binding site.

10. The method according to claim 9, wherein expression system is present in a cell-free environment.

11. The method according to claim 9, wherein said expression system is present inside of a cell.

12. The method according to claim 11, wherein said expression system comprises a TERT genomic sequence.

13. The method according to claim 9, wherein said repressing is by contacting said expression system with an agent that at least decreases the transcription repression activity of said TERT TF-8 and/or TF-13 repressor binding site.

14. The method according to claim 13, wherein said agent comprises a nucleic acid.

15. The method according to claim 13, wherein said agent comprises a peptide or a protein.

16. The method according to claim 13, wherein said agent is a small molecule.

17. A method for enhancing telomerase expression in a cell comprising a telomerase gene, said method comprising:
administering to said cell an effective amount of an agent that inhibits TERT transcription repression by a TF-8 and/or TF-13 repressor.

18. The method according to claim 17, wherein said administering is ex vivo.

19. The method according to claim 17, wherein said administering is in vivo.

20. A method for increasing the proliferative capacity of a cell, said method comprising:
administering to said cell an effective amount of an agent that inhibits TERT transcription repression by a TF-8 and/or TF-13 repressor.

21. The method according to claim 20, wherein said administering is ex vivo.

22. The method according to claim 20, wherein said administering is in vivo.

23. A method for delaying senescence in a cell, said method comprising:
administering to said cell an effective amount of an agent that inhibits TERT transcription repression by a TF-8 and/or TF-13 repressor.

24. The method according to claim 23, wherein said administering is ex vivo.

25. The method according to claim 23, wherein said administering is in vivo.

26. A method of determining whether an agent inhibits TF-8 or TF-13 repression of TERT transcription, said method comprising:
(a) contacting said agent with an expression system comprising a TERT TF-8 or TF-13 repressor binding site and a coding sequence operably linked to a TERT promoter under conditions such that in the absence of said agent transcription of said coding sequence is repressed;
(b) determining whether transcription of said coding sequence is repressed in the presence of said agent; and
(c) identifying said agent as an agent inhibits TF-8 or TF-13 repression of TERT transcription if transcription of said coding sequence is not repressed in the presence of said agent.

27. The method according to claim 26, wherein said contacting step occurs in a cell-free environment.

28. The method according to claim 26, wherein said contacting step occurs in a cell.

29. The method according to claim 26, wherein said agent is a small molecule.

30. An agent identified according to the method of claim 26.

31. A pharmaceutical composition comprising an agent according to claim 30.

32. A method for extending the lifespan of a mammal, said method comprising:
administering to said mammal an effective amount of an agent that inhibits TF-8 and/or TF-13 repression of TERT transcription.

33. The method according to claim 32, wherein said administering is ex vivo.

34. The method according to claim 32, wherein said administering is in vivo.

35. The method according to claim 32, wherein said mammal is a human.

36. A mammalian cell comprising a telomerase gene modified by deletion of any of the nucleotides found in a TERT TF-8 and/or TF-13 binding site.

37. A mammalian cell comprising a telomerase gene modified by an exogenous molecule bound to any of the nucleotides of the TERT TF-8 or TF-13 repressor binding site.

38. A method of producing a mammalian antibody, comprising the steps of:
isolating a B cell from a mammal, which B cell or its progeny cell is characterized by producing an antibody of interest;
immortalizing the B cell or its progeny by disrupting the natural function of its telomerase gene at any of its nucleotides in its TERT TF-8 or TF-13 repressor binding site; and
growing the immortalized B cell and its progeny under conditions which allow the cells to produce the antibody of interest.

39. The method of claim 38, further comprising:
separating away the antibody of interest from the B cell and its progeny.

40. A double stranded DNA decoy sequence consisting essentially of an isolated sequence of the TERT TF-8 or TF-13 repressor binding site.

41. A method of treatment comprising administering to cells the decoy sequence of claim 40.
